(19) United States
(12) Patent Application Publication
(10) Pub. No.: US 2017/0106161 A1
(43) Pub. Date: Apr. 20, 2017

(54) BREATHING MASK AND GAS PROVIDING DEVICE

(71) Applicant: Hsin-Yung Lin, Shanghai (CN)

(72) Inventor: Hsin-Yung Lin, Shanghai (CN)

(21) Appl. No.: 15/297,344

(22) Filed: Oct. 19, 2016

(30) Foreign Application Priority Data
Oct. 19, 2015 (CN) 201520807285X

(51) Int. Cl. A61M 16/06; A61M 16/20; A61M 16/14; A61M 16/08

(52) U.S. Cl. CPC A61M 16/0683; A61M 16/0875; A61M 16/208; A61M 16/14; A61M 2202/0208

(57) ABSTRACT

A breathing mask, capable of connecting a gas generator for providing gas generated by the gas generator to a user, includes a main body, a gas inputting tube, a one-way valve, and at least one gas exiting tube, wherein the main body further includes a containing part which forms a containing space for containing the nose of the user. The gas inputting tube is connected to the gas generator for providing the gas to the containing space. The one-way valve allows the gas to pass from the gas input tube to the containing space but blocks the gas from the containing space to the gas input tube. The gas exiting tube enables the gas to exit out from the containing space. The user can breathe in the gas generated by the gas generator from the containing space, and the waste air can exit through the gas exiting tube.

### PRIORITY CLAIM

This application claims the benefit of China Patent Application No. 201520807285.X filed on Oct. 19, 2015. The contents of the foregoing application are incorporated by reference in their entirety as if fully set forth herein.

### FIELD OF THE INVENTION

The present invention relates to a breathing mask and a gas providing device, and more particularly, the present invention relates to the breathing mask and the gas providing device, which can allow user to breath in specified gas smoothly.

### DESCRIPTION OF THE PRIOR ART

Breathing system is one of the several kinds supporting system for maintaining the patient's life in the hospital. A breathing mask is usually used to provide a high concentration of oxygen or cleaning air for patients. It can also be used to let respiratory failure patients breathe in the needed oxygen normally. In common, a breathing system includes a breathing mask and a gas tube. The gas tube connects the breathing mask and a gas generator, so that the oxygen or air generated by the gas generator can be entered to the breathing mask. The breathing mask can fit the patient's nose and mouth in order to provide high concentration of oxygen or air to the patients. The breathing system can also be connected to other medical gas or anesthetic gas generators for providing medical or anesthetic proposes.

Except for medical purpose, the breathing system can also be used for other purposes, such as providing the air in emergency or daily care. It can be achieved for any purposes of inhaling gas by nose or mouth when using the breathing system.

For example, the gas generator can generate hydrogen or oxygen for providing the medical gas sources. A free radical is produced by the oxidation of metabolism. It has one or many non-pairs of electrons or ions, which can attract the nearby electrons because of its active property. Furthermore, the free radical is created for maintaining the human body's functions by generating good chemical matters or resisting the diseases, such as generating enzymes and engulfing diseases by leukocytes. In addition to the reasons mentioned above, the free radical can be generated by outside environment such as smoke, drinking, radiation, UV, and electromagnetic wave, etc. According to current research, the free radical is the major cause of chronic diseases, cancers, and ageing.

Free radicals can be exhausted from the human body by composing the hydrogen to H2O. Hydrogen is a strong reduction of antioxidant. Furthermore, hydrogen can reduce free radicals from human body, which returns the acidity to alkalinity physique to achieve the effects of anti-oxidation, anti-ageing, the elimination of chronic diseases, and health care. Much research shows that the patient who has lung injury due to breathing high concentration of oxygen can be relieved by breathing hydrogen.

In the prior art, the device that can generate hydrogen or a mixture of hydrogen and oxygen is used as a common device for health protection. In general, the hydrogen or the mixture of hydrogen and oxygen which generated by a gas generator can enter a breathing mask through a gas inputting tube. The user wears the breathing mask to breathe in the hydrogen or the mixture of hydrogen and oxygen. The breathing mask is fitted to the user's face, and it can cover the nose, even the mouth. However, the user not only inhales the gas but also exhales the waste gas. The breathing mask is half-airtight to cover the nose so that the waste gas remains inside the breathing mask and blends with the hydrogen or the mixture of hydrogen and oxygen generated by the gas generator. Accordingly, it reduces the efficiency of the gas generator when the waste gas reacts with the mixture of hydrogen and oxygen and then becomes useless gas.

Therefore, it is necessary to develop a breathing mask to prevent the waste gas exhaled by users from interfering with the healthy gas which is supposed to be inhaled by users.

### SUMMARY OF THE INVENTION

The present invention proposes a breathing mask. According to one aspect of the invention, the breathing mask comprises a main body, a gas inputting tube, a one-way valve, and at least one gas existing tube. The main body has a containing part and a fitting part. The fitting part is connected to the containing part and is fit to the face of the user. The containing part forms a containing space for containing the nose of the user. The gas inputting tube is connected between the containing part and a gas generator. The one-way valve is disposed between the gas inputting tube and the main body. The one-way valve allows a generated gas from the gas generator to pass from the gas inputting tube to the containing space and block a space gas in the containing space into the gas inputting tube. The gas existing tube is connected between the containing part and the outside for exhausting the space gas in the containing space from the containing space to the outside.

According to an embodiment of the invention, a user can use the breathing mask connected to gas inputting tube while using gas generator. The user can wear the breathing mask which fits the face. The breathing mask can cover the nose in the position of central of the breathing mask. The generated gas from gas generator is entered to the containing space when the user breathes. In contrast, when the user exhales, the waste gas is entered into the containing space and then is exhausted to the outside by the gas exiting tube. The one-way valve is disposed between the gas inputting tube and the containing part so that the waste gas is entered to the gas exiting tube instead of entering to the gas inputting tube. Therefore, the waste gas doesn't stay in the containing space or the gas inputting tube to interfere with the generated gas.

According to another embodiment of the invention, the breathing mask further comprises the elements as mentioned before, wherein the gas inputting tube is rotatably connected to the containing part.

According to another embodiment of the invention, the breathing mask further comprises the elements as mentioned before, wherein the plurality of through holes are formed on the at least one gas existing tube.

According to another embodiment of the invention, the breathing mask further comprises the elements as mentioned before, wherein the containing space is formed by a central hollow of the containing part, and the fitting part is connected to an outer edge of the containing part.

According to another embodiment of the invention, the breathing mask further comprises the elements as mentioned above, wherein the gas inputting tube is connected to a central portion of the containing part, and the at least one gas exiting tube is connected to a side of the containing part.

According to another embodiment of the invention, the present invention proposes a gas providing device. According to one aspect of the invention, the gas providing device comprises a gas generator and a breathing mask. The breathing mask comprises a main body, a gas inputting tube, a one-way valve, and at least one gas existing tube. The main body has a containing part and a fitting part. The fitting part is connected to the containing part and fitted to a face of a user. The containing part forms a containing space for containing a nose of the user. The gas inputting tube is connected between the containing part and a gas generator. The one-way valve is disposed between the gas inputting tube and the main body. The one-way valve allows a generated gas from the gas generator to pass from the gas inputting tube to the containing space and block a space gas in the containing space from entering into the gas inputting tube. The gas existing tube is connected between the containing part and an outside for exhausting the space gas in the containing space from the containing space to the outside.

According to another embodiment of the invention, the gas providing device further comprises the elements as mentioned above, wherein the generated gas from the gas generator is a mixture of hydrogen and oxygen.

According to another embodiment of the invention, the gas providing device further comprises the elements as mentioned above, the gas providing device further comprises a vaporized gas generator. The gas inputting tube is connected between the vaporized gas generator and the containing part for inputting a vaporized gas into the containing space of the containing part.

According to another embodiment of the invention, the breathing mask further comprises the elements as mentioned above, wherein the plurality of through holes are formed on the at least one gas existing tube.

According to another embodiment of the invention, the breathing mask further comprises the elements as mentioned above, wherein the containing space is formed by a central hollow of the containing part, and the fitting part is connected to an outer edge of the containing part.

Many other advantages and features of the present invention will be further understood by the following detailed description and the appended drawings.

### BRIEF DESCRIPTION OF THE APPENDED DRAWINGS

FIG. 1A is a schematic diagram of a breathing mask according to an embodiment of the invention.

FIG. 1B is a schematic diagram of side view for a breathing mask according to FIG. 1A.

FIG. 2 is a schematic diagram of a gas providing device according to an embodiment of the invention.

FIG. 3 is a schematic diagram of a gas providing device according to another embodiment of the invention.

### DETAILED DESCRIPTION OF THE INVENTION

A detailed description of the hereinafter described embodiments of the disclosed apparatus and method are presented herein by way of exemplification and not limitation with reference to the Figures. Although certain embodiments are shown and described in detail, it should be understood that various changes and modifications may be made without departing from the scope of the appended claims. The scope of the present invention will in no way be limited to the number of constituting components, the materials thereof, the shapes thereof, the relative arrangement thereof, etc., and are disclosed simply as an example of embodiments of the present invention.

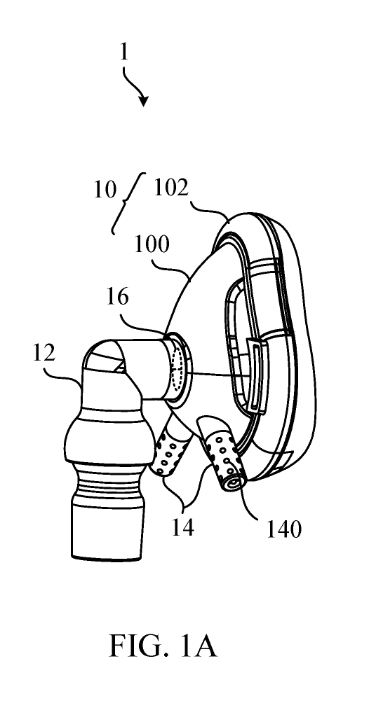

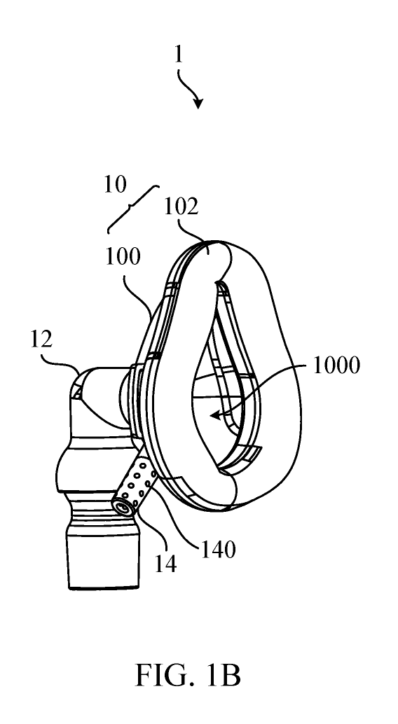

The present invention provides a breathing mask 1. Please refer to FIG. 1A and FIG. 1B. FIG. 1A is a schematic diagram of the breathing mask 1 according to an embodiment of the invention. FIG. 1B is a schematic diagram of side view for the breathing mask 1 according to FIG. 1A. The breathing mask 1 comprises a main body 10, a gas inputting tube 12, a one-way valve 16, and at least one gas existing tube 14. The main body 10 has a containing part 100 and a fitting part 102. The containing space 1000 is formed by a central hollow of the containing part 100, and the fitting part 102 is connected to an outer edge of the containing part 100. In fact, when user uses the breathing mask 1, the main body 10 is worn and let the fitting part 102 fit to the user's face. Then the nose, even the mouth is contained in the containing space 1000. Besides the main body of the breathing mask further comprises a headband which is disposed to the fitting part or the containing part so as to fix the main body of the breathing mask to the user's face. The gas inputting tube 12 is connected the central of the containing part 100 of the main body. In more detail, the gas inputting tube 12 is connected to another side of the containing space 1000 of the containing part 100.

The gas inputting tube 12 is connected between the containing part 100 and the gas generator. In fact, the gas generator is a device which can provide any kinds of medical or health care gas such as oxygen, hydrogen, mixture of hydrogen and oxygen, or any devices for providing anesthesia gas. The generated gas can be entered to containing space 1000 of containing part 100 from the gas inputting tube 12, and breathed by users from containing space 1000. Containing space 1000 forms an airtight space because the containing part 100 and the fitting part 102 fit the user's face, which can avoid the outside gas interfering with the generated gas from the gas generator.

The one-way valve 16 is disposed between the gas inputting tube 12 and the containing part 100. The one-way valve 16 can let the gas pass through in only one direction. The one-way valve 16 can make the gas generated by gas generator enter into the containing space 1000 for the user's breathing. Contrarily, the waste gas which the user exhales is blocked by the one-way valve 16 so that the waste gas cannot be entered into the gas inputting tube 12 from the containing space 1000.

The gas existing tube 14 is connected to the other side of containing space 1000 of containing part 100, and connected to the containing space 1000 which is located on the other side of containing part 100. As mentioned before, although the containing part 100 and the fitting part 102 can block most of the outside gas from entering into the containing space 1000, the waste gas can interfere with the gas which is exhaled by the user himself/herself in the containing space 1000. However, due to the one-way valve 16, the waste gas that the user exhales doesn't enter into the gas inputting tube 14 from containing space 1000. The waste gas can be entered into the gas exiting tube 14 from the containing space 1000 and be exhausted to the outside as well. In more detail, the waste gas that the user exhales is passed to the containing space 1000 to the outside through the gas exiting tube 14, so that the interference with the gas generated by the gas generator is reduced.

In fact, the gas exiting tube 14 can be various types to exhaust the waste gas and keep the generated gas from the gas generator. For example, as shown in FIG. 1A, the gas exiting tube 14 connects and extends to the bottom of the main body 10. The waste gas (no matter exhaled from the nose or the mouth) can be exhausted directly by the gas exiting tube 14 because the nostril direction and the mouth position are below to the gas exiting tube 14. In the other hand, the waste gas is carbon dioxide which is much heavier than oxygen, hydrogen, and air. The gas exiting tube 14 is positioned to the bottom of the main body 10 and extends downward, which can help the waste gas sink spontaneously.

According to an embodiment of the invention, there are two gas exiting tube 14, and they connect to the both sides of the lower part of the main body 10 so as to correspond to the nose position. However, there is not limited to two gas exiting tube 14. At least one gas exiting tube can achieve the waste gas exhausting effect. Moreover, a plurality of through holes 140 are formed on the gas exiting tube 14 for exhausting the waste gas effectively from the containing space 1000.

According to an embodiment of the invention, the breathing mask can provide the needed gas to the user without the interference of the outside gas. And, since the waste gas that users exhale themselves is blocked by the one-way valve, the waste gas cannot be entered into the gas inputting tube but be exhausted from the gas exiting tube. Accordingly, the gas exiting tube doesn't affect the generated gas from gas generator. Moreover, the gas exiting tube can increase the efficacy of the user's gas breathing.

In addition, when a user wears the breathing mask, he/she can keep various postures, such as lying on the bed or sitting on the chair. For users' comfort and the adaptation of the breathing mask, the gas inputting tube is rotatably connected to the containing part in order to fit the user's nose under different postures.

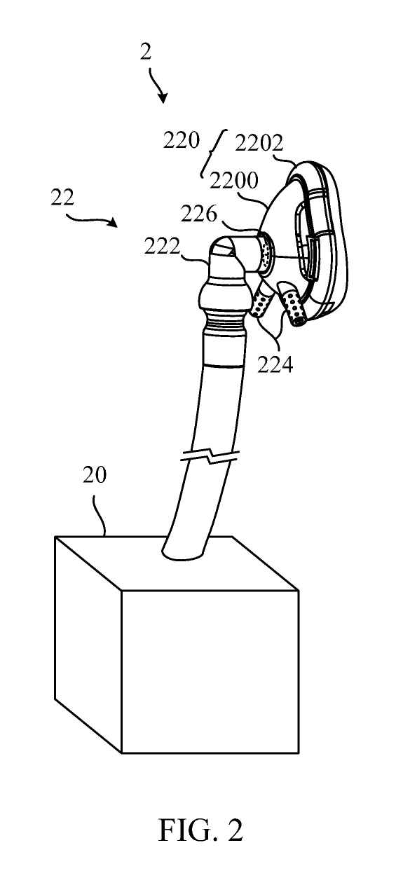

Please refer to FIG. 2. FIG. 2 is a schematic diagram of the gas providing device 2 according to an embodiment of the invention. As FIG. 2 shown, the gas providing device 2 comprises a gas generator 20 and a breathing mask 22. The gas generator 20 can generate the gas for users' breathing, such as hydrogen, oxygen, mixture hydrogen and oxygen, or anesthesia gas, etc. The breathing mask 22 comprises a main body 220, a gas inputting tube 222, a one-way valve 226, and at least one gas existing tube 224. The main body 220 has a containing part 2200 and a fitting part 2202. The gas inputting tube 222 is connected the central of the containing part 2200 and the gas generator 20, which provide the generated gas from gas generator 20 to the containing space of containing part 2200. The one-way valve 226 is disposed between the gas inputting tube 222 and the containing part 2200. The one-way valve 226 can let the gas pass through from gas generator 20 to the containing part 2200 in one direction. Also, at least one gas exiting tube 224 connects to the containing part 2200. There is substantially the same about the elements of the breathing mask 22 according to an embodiment of the invention; for example, a plurality of through holes are formed on the gas exiting tube 224, the containing space is formed by a central hollow of the containing part 2200, the fitting part 2202 is connected to an outer edge of the containing part 2200, etc.

When a user uses the gas providing device 2, the breathing mask 22 is worn on the face so that the containing part 2200 can cover the user's nose. In fact, the breathing mask 22 further comprises a headband so as to fix the breathing mask 22 to the user's face. However, any kinds of stuff that can fix the breathing mask to the user's face are reasonably chosen to use.

The gas generator 20 is started when a user is ready to wear the breathing mask 22. The generated gas from the gas generator 20 can be breathed by the user from gas generator 20, which is passed through from gas inputting tube 222 to the containing space of the containing part 2200. Due to the one-way valve 226, the waste gas that users exhale doesn't enter into the gas inputting tube 224 from the containing space. The waste gas is entered to the gas exiting tube 114 from the containing space and is exhausted to the outside. According to an embodiment of the invention, the breathing mask 22 can provide the generated gas by gas generator 20 to the user without the interference of the outside gas. Accordingly, the gas exiting tube doesn't affect the generated gas from gas generator. Moreover, the gas providing device can increase the efficacy of the user's gas breathing.

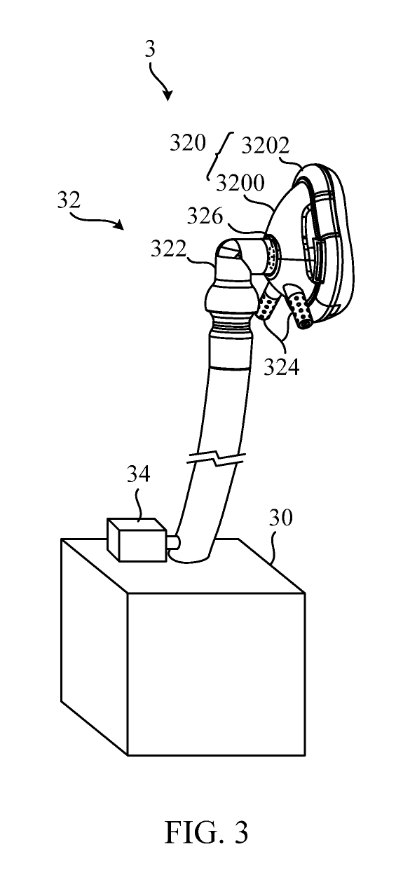

Please refer to FIG. 3; FIG. 3 is a schematic diagram of the gas providing device according to another embodiment of the invention. As FIG. 3 shown, the gas providing device 3 further comprises a vaporized gas generator 34. The gas providing device 3 is connected to a containing part 3200 through a gas inputting tube 322. The vaporized gas generator 34 can vaporize the gas and enters into the containing part 3200 through the gas inputting tube 322. In detail, the vaporized gas generated by the vaporized gas generator 34 is mixed with the generated gas (ex: hydrogen or mixture of hydrogen and oxygen) by the gas generator, and then is entered to the containing part 3200. Both effects of the vaporized gas and the hydrogen can be obtained by breathing these two gases. Please note that the other elements of the gas providing device 3 are substantially similar to the gas providing device 2.

According to an embodiment of the invention, the vaporized gas and the mixture of hydrogen and oxygen are mixed before entering the gas inputting tube 322. However, the vaporized gas and the mixture of hydrogen and oxygen can be separated to enter to the containing space. For example, the vaporized gas and the mixture of hydrogen and oxygen can be entered into the first gas inputting tube and the second gas inputting tube respectively. Then the two gases are mixed when entering to the containing space.

According to an embodiment of the invention, the breathing mask of gas providing device can provide the needed gas to the user without the interference of the outside gas. And the waste gas cannot be entered into the gas inputting tube and can be exhausted from the gas exiting tube. Moreover, the gas exiting tube can increase the efficacy of the users' gas breathing.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the meets and bounds of the appended claims.

The embodiments of the invention in which an exclusive property or privilege is claimed are defined as follows:

What is claimed is:

1. A breathing mask, comprising:
a main body having a containing part and a fitting part, the fitting part connected to the containing part and capable to be attached to a face of a user, and the containing part comprising a containing space for accommodating a nose of the user;
a gas inputting tube connected to the containing part;
a one-way valve disposed between the gas inputting tube and the main body for allowing a generated gas of a gas generator to pass unilaterally from the gas inputting tube to the containing space via the gas inputting tube; and
at least one gas existing tube connected to the containing part for exhausting an existed gas in the containing space.

2. The breathing mask of claim 1, wherein the gas inputting tube is rotatably connected to the containing part.

3. The breathing mask of claim 1, wherein a plurality of through holes are formed on the at least one gas existing tube.

4. The breathing mask of claim 1, wherein the fitting part is connected to an outer edge of the containing part.

5. The breathing mask of claim 1, wherein the gas inputting tube is connected to a central portion of the containing part, and the at least one gas exiting tube is connected to a side portion of the containing part.

6. A gas providing device, comprising:
a gas generator;
a main body having a containing part and a fitting part, the fitting part connected to the containing part and capable to be attached to a face of a user, and the containing part comprising a containing space for accommodating a nose of the user;
a gas inputting tube connected to the containing part;
a one-way valve disposed between the gas inputting tube and the main body for allowing a generated gas of the gas generator to pass unilaterally from the gas inputting tube to the containing space via the gas inputting tube; and
at least one gas existing tube connected to the containing part for exhausting an existed gas in the containing space.

7. The gas providing device of claim 6, wherein the generated gas of the gas generator is a mixture of hydrogen and oxygen.

8. The gas providing device of claim 6, further comprising a vaporized gas generator, the gas inputting tube connected between the vaporized gas generator and the containing part for inputting a vaporized gas into the containing space of the containing part.

9. The gas providing device of claim 6, wherein a plurality of through holes are formed on the at least one gas existing tube.

10. The gas providing device of claim 6, wherein the fitting part is connected to an outer edge of the containing part.
